(19) United States
(12) Patent Application Publication
(10) Pub. No.: US 2022/0241304 A1
(43) Pub. Date: Aug. 4, 2022

(54) SUBLINGUAL DELIVERY OF ANTI-VIRAL AGENTS AND CB2 RECEPTOR AGONISTS

(71) Applicant: Katherine Withers Hess, Toronto (CA)

(72) Inventor: Katherine Withers Hess, Toronto (CA)

(21) Appl. No.: 17/590,331

(22) Filed: Feb. 1, 2022

Related U.S. Application Data
(60) Provisional application No. 63/144,240, filed on Feb. 1, 2021.

(51) Int. Cl. A61K 31/704; A61K 31/397; A61K 31/05; A61K 9/00

(52) U.S. Cl. CPC A61K 31/704; A61K 9/0053; A61K 31/05; A61K 31/397

(57) ABSTRACT

A device is provided for treating inflammation, viral infection, and infectious disease conditions. The device includes a sublingual delivery film, dissolvable powder, compressed tablet, or tincture, with an ACE2 receptor binding competitor and a CB2 receptor agonist that could include cannabichromene. A composition is also provided to treat inflammation, viral infection, or infectious disease. The composition includes an ACE2 receptor binding competitor and a CB2 receptor agonist. A method is also provided for treating inflammation, viral infection, or infectious disease, including applying a sublingual delivery film, dissolvable powder, compressed tablet, or tincture, with an ACE2 receptor binding competitor and a CB2 receptor agonist. A composition for treating a patient with inflammation, viral infection and/or infectious disease is also provided, including an ACE2 receptor binding competitor and a CB2 receptor agonist.

The instant application claims priority as a non-provisional to U.S. application 63/144,240 filed on Feb. 1, 2020, the contents of which are hereby incorporated by reference.

### TECHNICAL FIELD

The present disclosure generally relates to the treatment of viral infection and, more specifically, to the sublingual application of virus and inflammation inhibitors.

### BACKGROUND

Effective treatments of viral infections and infectious diseases are an ongoing goal of healthcare to alleviate the potentially devastating outcomes of virus outbreaks. Although many synthetic compounds have been developed for the medical treatment of viral infections, the use of natural substances is an attractive approach since known compounds may have a more rapid approval for use in treatment and a smaller risk of side effects.

What is lacking in the field is a device and method for rapid and simple delivery of effective natural compounds to alleviate inflammation associated with infectious disease and to reduce viral infection.

### SUMMARY

The present disclosure describes a device, method, composition, and use thereof for the treatment of subjects having inflammation, viral infection and/or infectious disease.

In a first aspect of the present invention, there is provided a device for the treatment of inflammation, viral infection and/or infectious disease, including a sublingual delivery film wherein the film includes an Angiotensin-Converting Enzyme 2 (ACE2) receptor binding competitor and/or a CB2 receptor agonist.

In a further aspect of the present invention, there is provided a method for the treatment of inflammation, viral infection and/or infectious disease; the method includes applying a sublingual delivery film, wherein the film includes at least one of an ACE2 receptor binding competitor and a CB2 receptor agonist.

In a further aspect of the present invention, there is provided a composition for the treatment of a subject having inflammation, viral infection and/or infectious disease, the composition including an ACE2 receptor binding competitor and a CB2 receptor agonist.

In a further aspect of the present invention, there is provided a use of an ACE2 receptor binding competitor and a CB2 receptor agonist for the preparation of a medicament for the treatment of at least one of inflammation, viral infection, and infectious disease.

A more complete understanding of the disclosure can be obtained by reference to the following detailed description in connection with the attached drawings.

### BRIEF DESCRIPTION OF THE DRAWINGS

FIGS. 1A and 1B are schematic diagrams of anti-viral agents of the present invention.

FIG. 2 is a schematic diagram of inflammation suppression agents of the present invention.

FIG. 3A is a schematic diagram of an embodiment of a sublingual delivery device of the present invention for anti-viral agents.

FIG. 3B is a schematic diagram of a further embodiment of the sublingual delivery device of the present invention for anti-inflammatory agents.

The drawings provided herein are presented for convenience to explain the functions of the elements included in the described embodiments of the device and its method of use. Elements and details that are obvious to a person skilled in the art may not have been illustrated. Conceptual sketches have been used to illustrate elements that would be readily understood in the light of the present disclosure. Some details have been exaggerated for clarity. These drawings are not fabrication drawings and should not be scaled.

### DETAILED DESCRIPTION

The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

The following description refers to a device, method, composition, and use thereof for the treatment of inflammation, viral infection and/or infectious disease. It will be understood by those skilled in the art that the disclosure provided may be applied to other health conditions that benefit from reducing inflammation and/or inhibiting viral infection.

### ACE2 Receptor Binding Competition

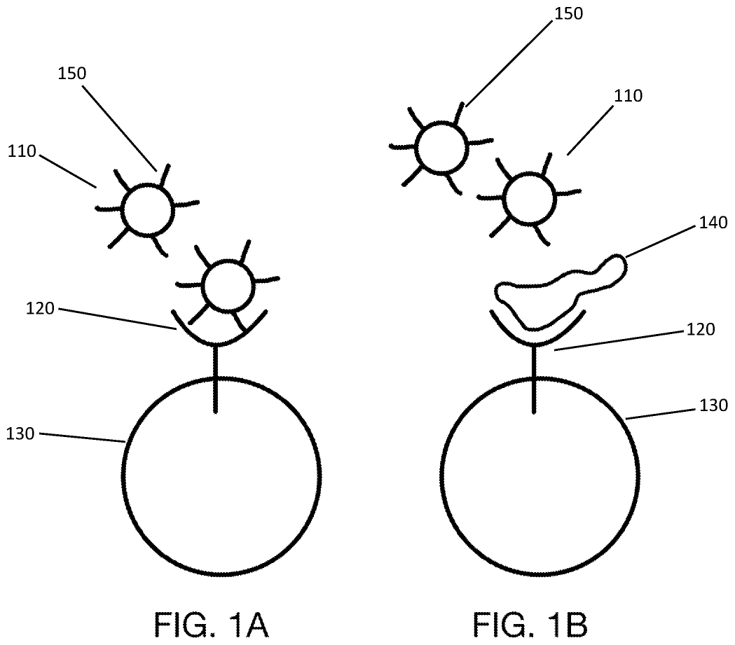

As illustrated in FIG. 1A, virus particles 110 bind to their target cell surface receptor proteins 120, which then are internalized by the cell 130. As illustrated in FIG. 1B, an approach to inhibit the uptake of the virus 110 into the cell 130 is to provide competing molecules 140 that compete with the virus for binding to the receptor 120. For example, an approach for inhibiting COVID-19 virus infection is to inhibit binding of the COVID-19 spike protein 150 to the ACE2 receptor 120, using competitors of ACE2 binding 140.

The COVID-19 spike protein 150, like other coronavirus spike proteins, extends from the virus particle 110 and is used for binding to target cell receptor proteins 120. For COVID-19, the target cell receptor protein 120 is ACE2, which is an enzyme that converts angiotensin I into angiotensin II, which in turn binds and activates angiotensin II receptor type 1 (AT1R). This activation leads to vasoconstrictive, proinflammatory, and pro-oxidative effects. ACE2 exists in two forms: a soluble form that represents circulating ACE2, and a structural transmembrane protein with an extracellular domain that serves as a receptor 120 for the COVID-19 spike protein 150. ACE2 exists both as a soluble protein and as a cell surface protein, the latter referred to as the ACE2 receptor. COVID-19 virus 110 uses the ACE2 receptor 120 for entry into target cells 130. Once the virus 110 is inside the target cell 130, the virus RNA genome is released into the target cells. After transcription and translation, the structural and non-structural proteins of COVID-19 and the RNA genome are further assembled into virions, which are transported through vesicles and released from target cells 130, releasing a multitude of virus particles that will continue to infect other target cells exponentially. Thus, by providing competing molecules 140 for binding of the virus particle 110 to the cell receptor protein 120, the propagation of the virus can be slowed and the viral infection can be curtailed.

Effective inhibitors of viral infection include ACE2 receptor binding competitors. Natural compounds that may be ACE2 receptor binding competitors include a triterpenoid saponin, for example, glycyrrhizic acid (GLR). Another ACE2 inhibitor may be nicotianamine (Muhseen et al. (2020) J Mol Liquids 320:114493; Muchtaridi. M et al. (2020) Molecules 25:3980).

### CB2 Receptor Agonists to Reduce Inflammation

Viral infections are also typically accompanied by inflammation. Inflammation is part of the body's immune response to harmful irritants such as infections, injuries, and toxins. Inflammation results from the release of antibodies and cytokines, such as tumor necrosis factor-α (TNF-α) and interleukin-6 (IL-6), and the recruitment of immune cells. Inflammation is accompanied by blood vessel dilation, resulting in redness, heat, and fluid accumulation in the inflamed tissue.

Although inflammation is a normal immune response and contributes to the immune reaction to fight disease, inflammation may also contribute to disease pathology. In the case of COVID-19, at least, inflammation may significantly contribute to disease mortality when the inflammation leads to damage of respiratory and other tissue. Thus, reduction of inflammation that accompanies viral infection is also a goal of the present disclosure. The present disclosure may also be applied to other instances where inflammation is present, for example, lung fibrosis, thrombosis in pulmonary small vessels, cardiovascular complications, rheumatoid arthritis, and atherosclerosis.

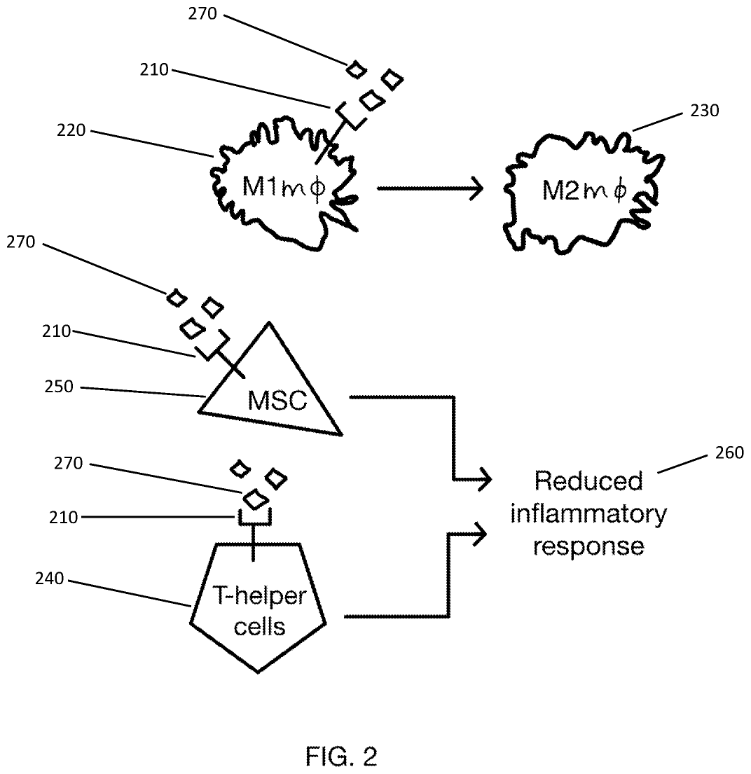

As illustrated in FIG. 2, a reduction of inflammation may be effected by activating the cannabinoid type-2 receptor (CB2R) 210. CB2R 210 is a G protein-coupled receptor that is highly expressed on immune cells, including macrophages 220, 230 and regulatory T cells 240, and mesenchymal stromal cells (MSC) 250. Activation of CB2R 210 on M1 proinflammatory macrophages 220 present in lung polarizes the M1 macrophages to M2 anti-inflammatory macrophages 230 and suppresses proinflammatory cytokines, including TNF-α and IL-6 (Staiano et al., J. Leukoc Biol. (2016) 99(4):531-540; Turcotte et al. Cell. Mol. Life Sci. (2016) 73:4449-4470; Kovalchuk et al., Aging (2021) 13:1571). Activation of CB2R 210 on MSC 250 and regulatory T cells 240 also leads to reduced inflammatory response 260 through changes in cytokine production and immune cell recruitment.

As further illustrated in FIG. 2. CB2R agonists 270 are ligands that activate CB2R 210. Thus, providing CB2R agonists 270 may reduce inflammation by activating CB2R 270 and inhibiting immune pathways that lead to macrophage activation and cytokine production. Effective agonists 270 for activation of CB2R 210 to suppress hyperimmune inflammatory responses include phytocannabinoids, endogenous cannabinoids, and terpenes. Examples of effective CB2R agonists 270 that target CB2 receptors 210 include cannabichromene (CBC), and the terpene beta-caryophyllene, along with many others that have documented measurements for CB2R affinity.

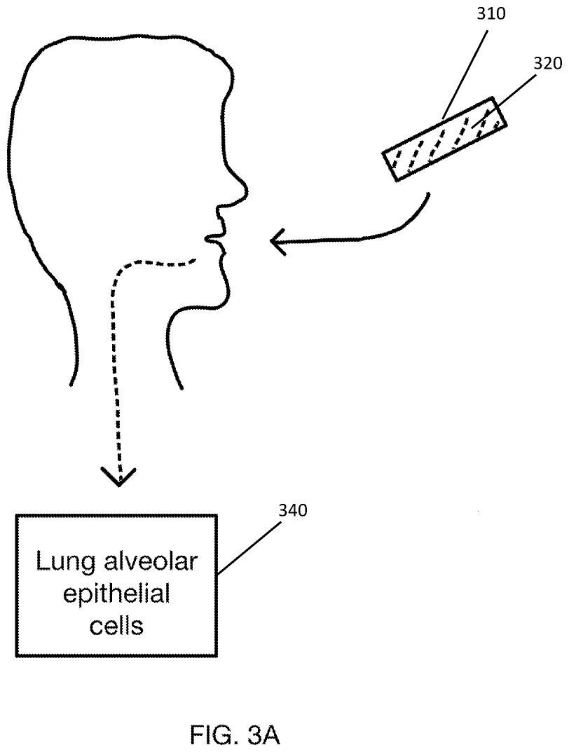

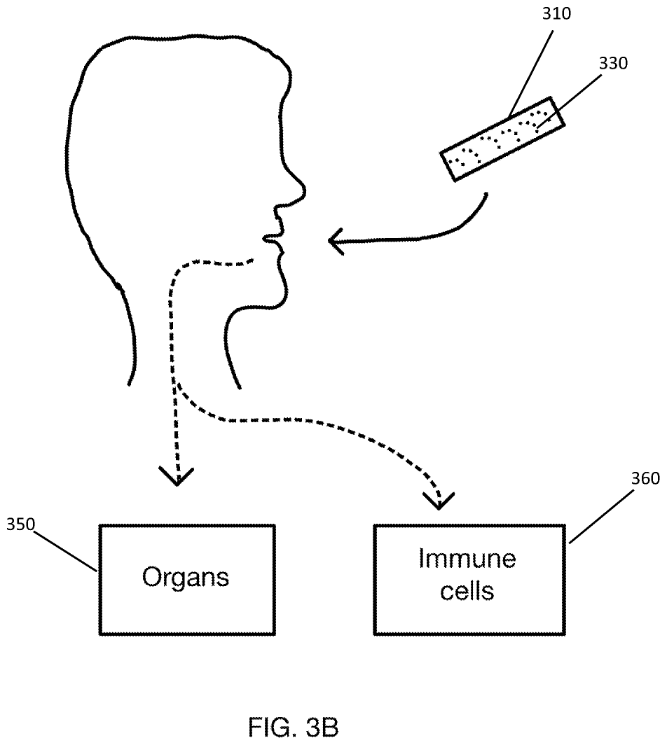

Referring to FIGS. 3A and 3B, a schematic of an embodiment of the present disclosure is provided. To address aspects of inflammation, viral infection and/or infectious disease, the present disclosure provides a device for rapid uptake of competitors of virus binding and inhibitors of inflammation. The device is a sublingual delivery film 310 for oral administration providing adsorption across the buccal or sublingual mucosa, which have abundant blood supply and high permeability of small molecules to the bloodstream. As provided by WO 2020/014776 and US publication 2017/0290870 A1, a sublingual delivery film 310 is a mucosally dissolvable, oral dispersible film with a consistent concentration of active ingredient and stable dispersion. Active ingredients 320, 330 may be layered onto or added into a structural matrix of the film strip 310. If the active ingredient 320, 330 is hydrophilic, it may be added to the water phase prior to dispersing with the oil-based mixture. If the active ingredient 320, 330 is oleophilic, it may be added with a subsequent oil-based mixture. The active ingredient 320, 330 may be infused or homogenously dispersed throughout the film composition. The sublingual delivery film 310 for treatment of inflammation, viral infection and/or infectious disease includes one or both of an ACE2 receptor binding competitor 320 and a CB2R agonist 330. The sublingual delivery film 310 may alternatively comprise a dissolvable powder, compressed tablet, or tincture.

As illustrated in FIG. 3A, the ACE2 receptor binding competitor 320, is adsorbed across the buccal or sublingual mucosa into the bloodstream and travels to lung alveolar epithelial cells 340. The ACE2 receptor binding competitor 320 competes with viral spike proteins (not shown) for binding to ACE2 receptor expressed on lung alveolar epithelial cells 340, thereby inhibiting viral infection. The ACE2 receptor binding competitor 320 may be a triterpenoid saponin or nicotianamine. An example of a triterpenoid saponin is glycyrrhizic acid (GLR).

As illustrated in FIG. 3B, the CB2R agonist 330 is adsorbed across the buccal or sublingual mucosa into the bloodstream and travels to organs 350, including liver, spleen, nasal epithelium, thymus, brain, lung, and kidney, where it binds to and activates CB2R. The CB2R agonist 330 also travels to immune cells, including macrophages, B cells. CD8+ and CD4+ T cells, natural killer T cells, monocytes, and neutrophils, and binds to the CB2R expressed on the immune cells, thereby suppressing the inflammatory response. The CB2R agonist 330 may be any of phytocannabinoids, endogenous cannabinoids, and terpenes. Other examples of effective CB2R agonists 330 include cannabichromene (CBC), and the terpene beta-caryophyllene, along with others that have documented measurements for CB2R affinity.

By combining an ACE2 receptor binding competitor 320 and a CB2R agonist 330, a targeted and synergistic treatment of viral infections such as COVID-19 is achieved by inhibiting cell infection by the virus and suppressing the destructive inflammatory response to the virus.

Referring further to FIGS. 3A and 3B, a method for the treatment of inflammation, viral infection and/or infectious disease is provided, using a sublingual delivery film 310 that includes active ingredients 320, 330. The method is oral administration of active ingredients 320, 330 by a sublingual delivery film 310, providing adsorption across the buccal or sublingual mucosa, which have abundant blood supply and high permeability of small molecules to the bloodstream. Active ingredients 320, 330 may be layered onto or added into a structural matrix of the film strip 310. The sublingual delivery film 310 for treatment of inflammation, viral infection and/or infectious disease includes one or both of an ACE2 receptor binding competitor 320 and a CB2 receptor agonist 330. Alternatively, oral administration may comprise a dissolvable powder, compressed tablet, or tincture. The ACE2 receptor binding competitor 320 may be a triterpenoid saponin or nicotianamine. An example of a triterpenoid saponin is glycyrrhizic acid (GLR). The CB2 receptor agonist 330 may be any of phytocannabinoids, endogenous cannabinoids, terpenes, cannabichromene (CBC), and the terpene beta-caryophyllene, along with others that have documented measurements for CB2R affinity.

A composition for the sublingual treatment of a subject having inflammation, viral infection and/or infectious disease is also provided. The composition includes one or both of an ACE2 receptor binding competitor 320 and a CB2R agonist 330. The ACE2 receptor binding competitor 320 may be a triterpenoid saponin or nicotianamine. An example of a triterpenoid saponin is glycyrrhizic acid (GLR). The CB2R agonist 330 may be any of phytocannabinoids, endogenous cannabinoids, terpenes, cannabichromene (CBC), and the terpene beta-caryophyllene, along with others that have documented measurements for CB2R affinity.

The use of an ACE2 receptor binding competitor 320 and a CB2R agonist 330 for preparation of a medicament for the sublingual treatment of inflammation, viral infection and/or infectious disease is also provided. The ACE2 receptor binding competitor 320 may be a triterpenoid saponin or nicotianamine. An example of a triterpenoid saponin is glycyrrhizic acid (GLR). The CB2R agonist 330 may be any of phytocannabinoids, endogenous cannabinoids, and terpenes, cannabichromene (CBC), and the terpene beta-caryophyllene, along with others that have documented measurements for CB2R affinity.

While embodiments of the device, method, composition, and use of a treatment for inflammation and/or viral infection have been illustrated in the accompanying drawings and described hereinabove, it will be appreciated by those skilled in the art that various modifications, alternate constructions, and equivalents may be employed. It should be further understood that the claims are not intended to be limited to the particular forms disclosed but rather to cover all modifications, equivalents, and alternatives falling within the spirit and scope of this disclosure.

Therefore, the above description and illustrations should not be construed as limiting the scope of the invention, which is defined in the appended claims.

What is claimed is:

1. A device for treatment of inflammation, viral infection, and infectious disease, the device comprising a sublingual delivery film, wherein the film includes at least one of:
an ACE2 receptor binding competitor; and
a CB2 receptor agonist.

2. The device as in claim 1, wherein the ACE2 receptor binding competitor comprises at least one of:
a triterpenoid saponin; and
nicotianamine.

3. The device as in claim 2, wherein the triterpenoid saponin comprises glycyrrhizic acid (GLR).

4. The device as in claim 1, wherein the CB2 receptor agonist comprises at least one of:
phytocannabinoids;
endogenous cannabinoids; and
terpenes.

5. The device of claim 1 wherein the sublingual delivery film comprises a dissolvable powder, a compressed tablet, or a tincture.

6. A method for treatment of inflammation, viral infection, and infectious disease, comprising applying a sublingual delivery film, wherein the film includes at least one of:
an ACE2 receptor binding competitor; and
a CB2 receptor agonist.

7. The method as in claim 6, wherein the ACE2 receptor binding competitor comprises at least one of:
a triterpenoid saponin; and
nicotianamine.

8. The method as in claim 7, wherein the triterpenoid saponin comprises GLR.

9. The method as in claim 6, wherein the CB2 receptor agonist comprises at least one of:
phytocannabinoids;
endogenous cannabinoids; and
terpenes.

10. The method as in claim 6, wherein applying a sublingual delivery film comprises applying a dissolvable powder, a compressed tablet, or a tincture.

11. A composition for sublingual treatment of a subject having inflammation, viral infection and infectious disease, comprising at least one of:
an ACE2 receptor binding competitor; and
a CB2 receptor agonist.

12. The composition as in claim 11, wherein the ACE2 receptor binding competitor comprises at least one of:
a triterpenoid saponin; and
nicotianamine.

13. The composition as in claim 12, wherein the triterpenoid saponin comprises GLR.

14. The composition as in claim 11, wherein the CB2 receptor agonist comprises at least one of:
phytocannabinoids;
endogenous cannabinoids; and
terpenes.

15. Use of an ACE2 receptor binding competitor and a CB2 receptor agonist for preparation of a medicament for treatment of at least one of:
inflammation;
viral infection; and
an infectious disease.

16. The use as in claim 15, wherein the ACE2 receptor binding competitor comprises at least one of:
a triterpenoid saponin; and
nicotianamine.

17. The use as in claim 16, wherein the triterpenoid saponin comprises glycyrrhizic acid (GLR).

18. The use as in claim 15, wherein the CB2 receptor agonist comprises at least one of:
phytocannabinoids;
endogenous cannabinoids; and
terpenes.

19. The use as in claim 15, wherein said medicament is delivered sublingually using a film.

20. The use as in claim 15, wherein said medicament is delivered using a dissolvable powder, compressed tablet, or tincture.
